(19) United States
(12) Patent Application Publication
(10) Pub. No.: US 2020/0016105 A1
(43) Pub. Date: Jan. 16, 2020

(54) COMPOSITIONS AND USES THEREOF FOR THE TREATMENT OF CHRONIC FELINE RESPIRATORY DISORDERS

(71) Applicant: OMNIPHARM DEVELOPMENTS LIMITED, DUBLIN (IE)

(72) Inventors: ALEXANDRA BECK, SAVIGNAC DE L'ISLE (FR); ELIZABETH HODGKINS, YORBA LINDA, CA (US); MICHAEL LAPPIN, FORT COLLINS, CO (US); VALARIE TYNES, SWEETWATER, TX (US)

(21) Appl. No.: 16/490,582

(22) PCT Filed: Mar. 1, 2018
(86) PCT No.: PCT/EP2018/055100

§ 371 Date: Sep. 2, 2019

Related U.S. Application Data
(60) Provisional application No. 62/466,604, filed on Mar. 3, 2017.

(51) Int. Cl. A61K 31/201; A61K 31/20; A61K 31/194; A61P 11/02

(52) U.S. Cl. CPC A61K 31/201; A61P 11/02; A61K 31/194; A61K 31/20

(57) ABSTRACT

The present invention relates to compositions useful in the field of veterinary medicine and more particularly compositions comprising pheromones for treating feline chronic respiratory disorders and complications thereof and/or feline chronic infectious disorders or conditions.

### FIELD OF THE INVENTION

The present invention relates to compositions useful in the field of veterinary medicine and more particularly compositions comprising pheromones for treating feline chronic respiratory disorders and complications thereof and/or feline chronic infectious disorders or conditions.

### BACKGROUND OF THE INVENTION

Feline infectious diseases are quite frequent. The main inducer of feline infectious diseases has been deemed to be viruses which infect feline airways, and felines infected with viruses mostly show common cold-like symptoms such as inflammation of superior airway, cough, sneezing, conjunctivitis, rhinorrhea, nasal discharge, drooling, fever, pneumonia, stomatitis, glossitis, granular adenitis, lingual ulcer, ulcer of nasal end, systematic skin ulcer, turbinal ulcer, scours, vomiting, anorexia and reduction of vitality. More particularly, when felines are infected with feline herpesvirus, they become more likely to show the above-mentioned symptoms.

Feline herpesvirus 1 (FHV-1) is the most common feline infectious disease in cats with over 90% of cats housed in group settings being infected. The organism is associated with severe morbidity during the acute phase of infection (upper respiratory disease, conjunctivitis, and keratitis) and many infected cats develop chronic and recurrent clinical signs of disease.

Feline herpesvirus 1 (FHV-1) is believed to be the most common feline infectious disease in cats; infection can be subclinical, or it may result in clinical signs of disease including sneezing, cough, conjunctivitis, pyrexia, keratitis, dyspnea, inappetence, lethargy, and occasionally pneumonia and death. Morbidity and mortality in crowded or stressful environments such as shelters, can be high. Upper tract respiratory disorders include sneezing, cough, and/or conjunctivitis.

Following acute exposure, most cats develop persistent infection, with the trigeminal ganglia serving as the main site of viral latency. Reactivation of the latent FHV-1 can occur with recurrence of clinical signs and increases in FHV-1 shedding. Stressful events are believed to result in FHV-1 reactivation within the first weeks. In a study, the average time to development of signs of upper respiratory infections (URI) was 8.3 days, and those cats with the highest stress scores during the first week in the shelter were more likely to develop URI (Tanaka A, Wagner D C, Kass P H, et al. Associations among weight loss, stress, and upper respiratory tract infection in shelter cats. J Am Vet Med Assoc 2012; 240: 570-576).

At present, there exists no treatment worth mentioning for feline virus diseases as well as feline respiratory diseases, and actually these diseases are only treated with symptomatic treatments such as trophotherapy by administering to felines to be treated nutrient feeds in order to relieve to recover their physical vitality and/or their dehydration symptoms, as well as administering antibiotics to them to prevent multiple or secondary infections, and physically removing viscous secreta.

There is thus a need to develop new treatments for treating feline chronic respiratory disorders and complications thereof and/or feline infectious disorders or conditions, and in particular feline sneezing associated with infection.

Pheromone therapy, “pheromonatherapy”, or “pheromonotherapy” is a way to manage behavior problems in non-human mammals by using chemicals that have pheromonal properties. It is an innovative treatment of stress related problems for non-human mammals, including domestic animal species, more particularly cats and dogs, and non-companion animal species. The pheromonatherapy has been studied most extensively in dogs and cats.

Pheromones are chemical substances used for communication between individuals of the same species. Some chemicals that provide interspecies communication are called allelochemicals. Some compounds are known to be a pheromone in one species, but have been observed to have behavioral effects in other species. Pheromones will be used herein in a large sense, i.e. any naturally or non-naturally (synthetic) occurring compound that have a behavioral impact on non-human mammals. The precise mechanism of action of most pheromones is still unknown but they induce some modifications in both the limbic system and the hypothalamus.

Pheromonotherapy is a great way of managing non-human mammals welfare and more particularly some common stress-related behavioral disorders in domestic mammals, such as dogs and cats.

It has been discovered that a composition of pheromones can reduce clinical symptoms of feline chronic respiratory disorders due to infections. More particularly, it has been shown here that exposure to pheromones lessened sneezing associated with FHV-1 and increased relaxation during sleep period. The same composition of pheromones could help prevent recurrences or relapses of these respiratory disorders.

### SUMMARY OF THE INVENTION

The present invention therefore relates to a veterinary composition comprising pheromones for use in the treatment of chronic feline respiratory disorders or complications thereof and/or feline chronic infectious disorders or conditions.

The present invention also concerns a method for treating feline chronic respiratory disorders or complications thereof and/or feline chronic infectious disorders or complications comprising administering to a feline in need thereof a veterinary composition comprising pheromones.

The present invention further deals with a use of a veterinary composition of pheromones for the manufacture of a medicament for treating feline chronic respiratory disorders or complications thereof and/or feline chronic infectious disorders or conditions.

The present invention further concerns a use of a veterinary composition comprising pheromones for reducing clinical symptoms due to feline chronic infections, such as herpes infection, including FHV-1 infection. The present further concerns a use of a veterinary composition comprising pheromones for reducing clinical symptoms due to feline chronic respiratory disorders, particularly feline chronic upper respiratory tract disorders, due to infections, such as herpes infection, including FHV-1 infection.

### DESCRIPTION OF THE FIGURES

FIG. 1: Study timeline

FIG. 2: Proportions of observation points with sneezing by group and period.

* denotes a statistical difference between groups

### DETAILED DESCRIPTION OF THE INVENTION

In a particular embodiment, the invention relates to a veterinary composition comprising pheromones for use in the treatment of feline respiratory disorders, more specifically chronic upper respiratory tract disorders. More particularly, the feline chronic respiratory disorders are due to infections, such as herpes infection, including FHV-1 infection. More specifically, the invention provides a veterinary composition comprising pheromones particularly useful in the treatment of feline sneezing associated with infection, such as herpes infection, including FHV-1 infection.

In a more particular embodiment, the invention relates to a veterinary composition comprising pheromones for use in the treatment of clinical symptoms and/or prevention of recurrences or relapses of feline chronic respiratory disorders, such as sneezing, due to infections, more particularly due to herpes infection, including FHV-1 infection.

In another embodiment, the invention relates to a veterinary composition comprising pheromones for use in the treatment of clinical symptoms and/or prevent recurrences or relapses of feline chronic respiratory disorders, such as sneezing, due to infections, more particularly due to herpes infection, including FHV-1 infection.

A further object of the invention is a use of a veterinary composition comprising pheromones for reducing clinical symptoms due to feline chronic infections, such as herpes infection, including FHV-1 infection.

Another object of the invention is a use of a veterinary composition comprising pheromones for reducing clinical symptoms due to feline chronic respiratory disorders, particularly feline chronic upper respiratory tract disorders, due to infections, such as herpes infection, including FHV-1 infection.

The clinical symptoms of feline chronic respiratory disorders, which are generally due to FHV-1 infections, are more specifically inflammation of superior airway, cough, sneezing, conjunctivitis, rhinorrhea, nasal discharge or nasal congestion. It may also evolve towards drooling, ocular discharge, blepharospasm, fever, asthma, pneumonia, stomatitis, gastro-intestinal diseases, such as irritable bowel syndrome, glossitis, granular adenitis, lingual ulcer, ulcer of nasal end, systematic skin ulcer, turbinal ulcer, scours, vomiting, anorexia and/or reduction of vitality. The feline upper chronic respiratory tract disorders are generally inflammation of superior airway, cough, sneezing, conjunctivitis, rhinorrhea, nasal discharge, nasal congestion or a mixture thereof.

In a particular embodiment, clinical symptoms due to feline chronic respiratory disorders due to infections are cough, sneezing, nasal discharge, and/or nasal congestion, and is preferably sneezing.

The clinical symptoms of feline chronic infectious disorders generally infect feline airways, and include therefore inflammation of superior airway, cough, sneezing, conjunctivitis, rhinorrhea, nasal discharge or nasal congestion. The symptoms can also include drooling, ocular discharge, ocular congestion, blepharospasm, fever, asthma, pneumonia, stomatitis, gastro-intestinal diseases, such as irritable bowel syndrome, glossitis, granular adenitis, lingual ulcer, ulcer of nasal end, systematic skin ulcer, turbinal ulcer, scours, vomiting, anorexia and/or reduction of vitality.

In a particular embodiment, clinical symptoms due to feline chronic infections are cough, sneezing, nasal discharge, nasal congestion, ocular discharge, ocular congestion, and/or blepharospasm, and is preferably sneezing.

Recurrences or relapses of feline respiratory disorders due to infections, more specifically due to FHV-1, often occur and that is the reason why such disorders are considered chronic. These recurrences or relapses of feline respiratory disorders, and in particular sneezing due to infection, seem due to a lack of vitality, a lack of relaxation, a lack of immunodefense, and/or a lack of sleep. It has been found here that by administering composition comprising pheromones can prevent or reduce such recurrences or relapses, in particular by increasing relaxation during sleep period of felines.

In another embodiment, the invention relates to a veterinary composition comprising pheromones for use in the treatment of clinical symptoms and/or prevention recurrences or relapses of feline chronic respiratory disorders due to infections, more particularly due to herpes infection, including FHV-1 infection.

More preferably, the feline chronic upper respiratory tract disorders are generally inflammation of superior airway, cough, sneezing, conjunctivitis, rhinorrhea, nasal discharge, nasal congestion or a mixture thereof.

In another embodiment, the invention relates to a veterinary composition comprising pheromones for use in the treatment of feline upper respiratory tract disorders.

More particularly, the feline chronic respiratory disorders are due to infections, such as herpes infection, including FHV-1 infection. According to a specific embodiment, the invention relates to a veterinary composition comprising pheromones for use in the treatment of sneezing, more preferably sneezing associated with infection.

In a particular embodiment, the composition according to the invention lessens or reduces sneezing, and more particularly sneezing associated with infection, such as herpes virus, including more particularly FHV-1.

In another particular embodiment, the composition according to the invention increases relaxation evidenced by increased sleep period of felines with infectious disorders and/or respiratory disorders.

The present invention relates to composition or a mixture comprising pheromones. The said pheromones are one or more fatty acids and/or esters thereof or more specifically ethyl or methyl esters thereof, as active ingredients. The composition thus comprises of a fraction or mixture of esters of fatty acids or methyl ester derivatives of fatty acids. They can be administered to a feline through diffusion into ambient air.

The amounts of fatty acid, ester derivatives or methyl ester derivatives in the composition may vary in a wide range. They can range approximately between 0.5 and 95%, preferably between 1 and 70% or between 1;5 to 50% with respect to the total amount of the veterinary composition.

“Fatty acids” means according to the invention hydrocarbon chain with monocarboxylic or dicarboxylic acids, saturated or unsaturated, linear or branched, and active, and capable of inducing behavior changes in non-human mammals. These fatty acids generally have C4-C22. They are chosen among oleic acid, palmitic acid, azelaic acid, pimelic acid, capric acid, myristic acid, palmitoleic acid, linoleic acid, stearic acid, arachidonic acid, n-butyric acid, isobutyric acid, a-methylbutyric acid, capric acid, pivalic acid, y-linoleic acid, eicosapentanoic acid, pentadecanonic acid, tridecanoic acid or docosahexanoic acid.

“Derivatives of fatty acids” means all active, volatile derivatives of fatty acids. Preferably, the derivatives are in ester or methyl or ethyl ester forms.

The mixtures comprise at least one fatty acid such as oleic acid, derivatives of oleic acid, ester or methyl ester derivative thereof.

By way of examples, we can cite fatty acid mixtures, or their derivatives, including esters or methyl ester thereof, such as:
a mixture of oleic and palmitic acid;
a mixture of oleic and n-buytric acid;
a mixture of oleic acid, palmitic acid and linoleic acid;
a mixture of oleic acid, palmitic acid, linoleic acid and palmitoleic acid;
a mixture of capric acid, lauric acid, myristic acid, palmitoleic acid, palmitic acid, linoleic acid and oleic acid;
a mixture of oleic acid, palmitic acid, linoleic acid, myristic acid;
a mixture of oleic acid, palmitic acid, linoleic acid, lauric acid and myristic acid
a mixture of oleic acid, palmitic acid, linoleic acid, myristic acid and pentadecanonic acid;
a mixture of oleic acid, palmitic acid, linoleic acid, myristic acid, pentadecanonic acid and stearic acid;
a mixture of oleic acid, palmitic acid, linoleic acid, myristic acid, lauric acid and pentadecanonic acid;
a mixture of lauric acid, myristic acid, pentadecanonic, palmitic acid, stearic acid, oleic acid, linoleic acid; or more preferably
a mixture of oleic acid, azelaic acid, pimelic acid and palmitic acid.

The composition of the invention may comprise the above-mentioned fatty acids in appropriate proportions which are well known to any skilled person. By way of example, the mixtures can contain, with respect to the total weight of fatty acids and derivatives thereof:
about 55-65% of oleic acid and 45-35% of palmitic acid, their derivatives, and its ester or methyl ester derivatives;
about 45% of oleic acid, 16% of azelaic acid, 18% of pimelic acid, and 21% of palmitic acid, their derivatives, ester or methyl ester derivatives thereof;
about 30% of palmitic acid, 30% of oleic acid, and 40% linoleic acid, their derivatives, ester and methyl ester derivatives thereof; or
about 30% of palmitic acid, 40% of linoleic acid, 10% acid palmitoleic and 20% of oleic acid, their derivatives, ester and methyl ester derivatives thereof.

The percentages are expressed by weight, unless specified otherwise.

As used herein, the term “about” or “approximately” will be understood by a person of ordinary skill in the art and will vary to some extent on the context in which it is used. If there are uses of the term which are not clear to persons of ordinary skill in the art given the context in which it is used, “about” or “approximately” will mean up to plus or minus 10% of the particular term.

According to an embodiment of the present invention, the composition comprises at least a therapeutically effective amount of active fatty acids, derivatives thereof, including ester or methyl ester derivatives thereof, which are more particularly selected in the group consisting of oleic acid, azelaic acid, pimelic acid and palmitic acid. The mixture of fatty acids preferably comprises at least about 45-60% oleic acid, 6-10% of azelaic acid, 8-12% of pimelic acid, and 13-18% of palmitic acid, with respect to the total weight of fatty acids and derivatives thereof. In the case where ester and methyl ester forms are used, the mixture comprises between about 45-65% methyl oleate, between about 6-10% dimethyl azelate, between about 8-12% of dimethyl pimelate, and between about 13-18% of methyl palmitate, with respect to the total weight of fatty acids and derivatives thereof. Preferably, the mixture of esters of fatty acids consist approximately 47-51% of methyl oleate, approximately 7-9% of azelate of dimethyl, between 9-11% of dimethyl pimelate and approximately 14-16% of methyl palmitate, with respect to the total weight of fatty acids and derivatives thereof.

The compositions of the invention comprise pheromones as described above. The compositions can be devoid of solvent, excipients, vegetable extracts, or even aqueous phase. Alternatively, the compositions can comprise solvents, excipients, vegetable extracts. They can comprise solvents, including isoalkanes and cycloalkanes. They are usually present in the form of oily and liquid phases depending of the temperature and may be heated by any appropriate means so as to obtain a diffusion of fatty acids and their derivatives, ester or methyl ester derivatives thereof in the air.

Thereby, the fatty acid mixtures according to the invention, may be administered to felines by diffusion in the ambient air. The administration of felines can take place by heating or not. Diffusion of the composition in the ambient air from 6 to 30 consecutive days.

In a particular embodiment, the composition as disclosed herein is in a form intended for oral, nasal, intradermic, cutaneous, inhalation or parenteral administration. Preferably, the composition is in a form intended for diffusion in the ambient air.

In a further particular embodiment, the composition for use in the present invention is in a form of a liquid solution, suspension, solid or semi-solid, powders, pellets, capsules, granules, sugar coated pills, gelules, sprays, pills, tablets, pastes, implants or gels.

In ambient temperatures, the composition can be under a liquid form or under a solid form, which is a function of the length and the structure of compounds contained in the composition of the invention and in particular the solvents or excipients and pheromones, and therefore the length and the structure of the carbon chains of compounds.

Dispensers for diffusion can be selected by one skill in the art depending on the forms (solid or liquid) of the compositions. It can be for instance the dispenser described in US design patent U.S. D752,730S.

In a preferred embodiment, the administration is done by inhaling by felines, of the compositions of pheromones, which can be diffused in the ambient air around the feline's habitat.

Felines include all the members of the feline and feline family, domestic cats, and more commonly all races of cats. They include domestic, farm, or zoo felines, and in particular cats at any age, such as kittens.

Another object of the invention is a kit or an administrative device for the diffusion of the composition of the invention as defined above, to felines permitting their diffusion in the air.

These devices may be in particular, electric devices for vaporizing or diffusion comprising solid support having a ceramic plate and a polymer matrix on which sufficient quantities of the composition of the invention as defined above, are applied in a way that the diffusion can take place. These devices may be in the form of an electric pin fitted with a compartment consisting of the support ceramic plate that is suitable for heating and on which one can place a small plate impregnated with the composition of the fatty acids according to the invention. The device is made active by plugging to an electric source. The fraction of the composition of the invention is diffused under the effect of the heat through the openings in the compartments of the small plate. These are comparable to the ones used for diffusing anti mosquito repellents. The devices can also be the dispenser as described in US design patent U.S. D752,730S.

Further aspects and advantages of the invention will be disclosed in the following experimental section.

### EXAMPLES

### Example 1

### Preparation of Veterinary Composition

The veterinary composition (Feliway®) was prepared by mixing the following components as showed in the Table 1 below:

TABLE 1

| Compounds | g/100 g composition | pheromone fraction (%) |
|---|---|---|
| Dimethyl pimelate | 0.23 g/100 g | 11.558 |
| Dimethyl azelate | 0.18 g/100 g | 9.045 |
| Methyl oleate | 1.23 g/100 g | 61.809 |
| Methyl palmitate | 0.35 g/100 g | 17.588 |

QSP 100 g with Isopar V (solvent)

The used diffuser is as described in design patent U.S. D752′730 S

The diffuser can be used in a room having an area from 20 up to 70 m². The refill contains 48 ml Feliway product and will release the product in 20 to 30 days.

### Example 2

The study described here was designed to determine whether kittens with experimentally induced FHV-1 infections that were housed in equivalent rooms containing either a Feliway® diffuser or a Placebo diffuser and subjected to housing change induced stress would differ in behavioral scores, clinical scores, FHV-1 shedding, or serum cortisol concentrations.

### Materials and Methods

Cats. Six neutered male and six spayed female 5-month-old, purpose-bred kittens were used in this 12-week pilot study. Eight weeks prior to the study described here, each of the 12 kittens was infected with FHV-1 by intranasal instillation for a study in which the 12 kittens were the control group. Re-using these kittens for the purpose of this new study prevented from having to re-inoculate other cats experimentally. In that study, FHV-1 infection was confirmed in all kittens and each developed clinical signs consisting of fever, sneezing, ocular and/or nasal discharge, nasal congestion, conjunctivitis, and/or blepharospasm. At the time of this study, none were undergoing treatment for URI and occasional sneezing, serous nasal discharge, or serous ocular discharge were the only potential manifestations of FHV-1 present intermittently in the kittens. The study described here were approved by the Institutional Animal Care and Use Committee.

The kittens were randomized into a pheromone group (Feliway® diffuser) or placebo group (placebo diffuser); each Group consisted of three males and three females. The Groups were housed in two separate but similarly sized rooms (Pheromone group=8′6″ width×10′3″ length×9′ height; placebo group=9′7″ width×9′ length×9′ height) of the research facility which had two separate air exchangers. All kittens were provided dry food and water ad libitum and were provided identical perches and enrichment devices.

Clinical scoring. A clinical score sheet adapted from other FHV-1 vaccination or treatment studies was used in this study (Table 1). For some comparisons, a total clinical score was calculated for each kitten each day by adding the individual clinical score parameters recorded for that day. Body temperatures were estimated by microchip. Elevated body temperature was defined as >102.5° F. Heart and respiratory rates were measured daily when auscultation was not obscured by loud purring. Body weights were measured weekly.

TABLE 1
Clinical scoring rubric
Due to statistically low occurrence of scores >1, binomial analyses performed
using 0 or 1 to indicate presence or absence of clinical sign

| Clinical sign | Score |
|---|---|
| Conjunctivitis | 0 = None |
| | 1 = Mild |
| | 2 = Moderate |
| | 3 = Severe |
| Blepharospasm | 0 = None |
| | 1 = Eye < 25% closed |
| | 2 = Eye 25-50% closed |
| | 3 = Eye 50-75% closed |
| | 4 = Eye completely closed |
| Ocular discharge | 0 = None |
| | 1 = Mild serous (clear) discharge |
| | 2 = Moderate mucoid (white) discharge |
| | 3 = Severe mucopurulent (moist yellow-green) discharge |
| Body temperature | 0: ≤102.5 |
| (microchip) | 1: >102.5 |
| Cough | 0 = None |
| | 1 = Observed |
| Sneezing (yes/no) | 0 = None |
| | 1 = Observed |
| Nasal discharge | 0 = None |
| | 1 = Mild serous (clear) discharge |
| | 2 = Moderate mucoid (white) discharge |
| | 3 = Severe mucopurulent (moist yellow-green) discharge or hemorrhagic (bloody/red) |
| | discharge |
| Nasal congestion | 0 = None (no congestion present; able to breathe through both nares without difficulty) |
| (if score varies during | 1 = Mild/Minor congestion (barely audible; audible on close listening, subtle snoring |
| observation period, record | sounds on inhalation ANY time during the observation period |
| highest score observed) | 2 = Moderate congestion (easily audible; consistently audible throughout observation |
| | period; audible snoring sounds on inhalation or expiration that are likely to originate |
| | from the nasal cavity) |
| | 3 = Severe congestion (audible across the room, with or without open mouth breathing; |
| | minimal nasal air flow noted from one or both nares after local debris is cleared away) |

Behavioral scoring. A number of different behavioral assessment scales used in previous shelter and other studies were reviewed as tools to assess stress and behavior in the kittens. Based on observations of these kittens during the study in which they were inoculated with FHV-1 and the fact that the kittens studied herein were purpose-bred research kittens who had already been habituated to the research facility, housing, each other, and human interactions, a modified scale was designed (Table 2). The behavioral observation metrics were also designed to accommodate ease and efficiency and to not distract from FHV-1 clinical scoring. The rubric contained lists of typical feline postures, vocalizations, and actions which represented either normal, relaxed-calm, or stress-related behaviors which could be observed and objectively scored (Table 2). Due to the overall engaging personalities and temperaments of the particular group of kittens in this study, the rubric was further adapted before and during the equilibration period. The final rubric that was applied when the diffusers were introduced into the rooms contained 28 individual behaviors, recorded at five different specified time-points during the 45-minute scoring period per room each morning (Table 2). “Snapshot” observations were performed for 15 seconds per kitten at the following four time-points: upon entry into room (“SS1” time-point), during clinical scoring handling (“SS2” time-point), immediately following clinical scoring handling (“SS3” time-point), and at the 45-minute mark (“SS4” time-point). Behaviors were also recorded throughout the 20-30 minutes between SS3 and SS4 (“Long” time-point) (Table 2).

TABLE 2
“Long” time-point: While sitting down in room, approx 20-30 minutes, during time between SS3 and SS4

| | |
|---|---|
| Housing disarray: Litterbox overturned with litter, feces on | Y/N |
| floor during group housing. During cage housing: cage disarray - | (scored during Long time-point because more time allotted; however, |
| kibble, water, urine, litter scattered throughout cage | this housing disarray score represents the condition of room or cage |
| (litterboxes permanently affixed to cages so they cannot easily | upon scorers' entry into room) |
| be overturned by kitten) | |
| Diarrhea present? | Y/N |
| Urinated/Defecated (did you SEE kitten urinate/defecate) | Y/N |
| Eating/drinking observed? (did you SEE kittten eating or | Y/N |
| drinking) | |
| (Group housed only) Vocalization: Purring | Y/N (N also includes unknown - if not close enough to hear) |
| Vocalization: Meow | 0: no meows |
| (0, 1 during group housing) | 1: some occasional meows during period |
| (0, 1, 2 during cage housing) | 2: excessive meowing during pd (during cage housing) |
| Vocalization: Hiss/growl | Y/N |
| Any fighting/spats/aggression toward other cats or humans - | Y/N |
| present? (NOT PLAY) | |
| Hiding behavior | Y/N |
| Interacting/playing with objects or other cat(s) (or human | Y/N |
| during group housing) | |
| “Making biscuits” present? | Y/N |
| (Group housed only) Climbing on objects or on object | Y/N |
| (Group housed only) Climbing on person; or currently on | Y/N |
| person | |
| Groomed self | Y/N |
| (Group housed only) Groomed another cat (“allogrooming”) | Y/N |
| (Group housed only) Groomed person (licked person) | Y/N |
| (Cage housed only) Pawing through kennel | Y/N |
| (Cage housed only) Pacing (repetitive walking back and forth) | Y/N |

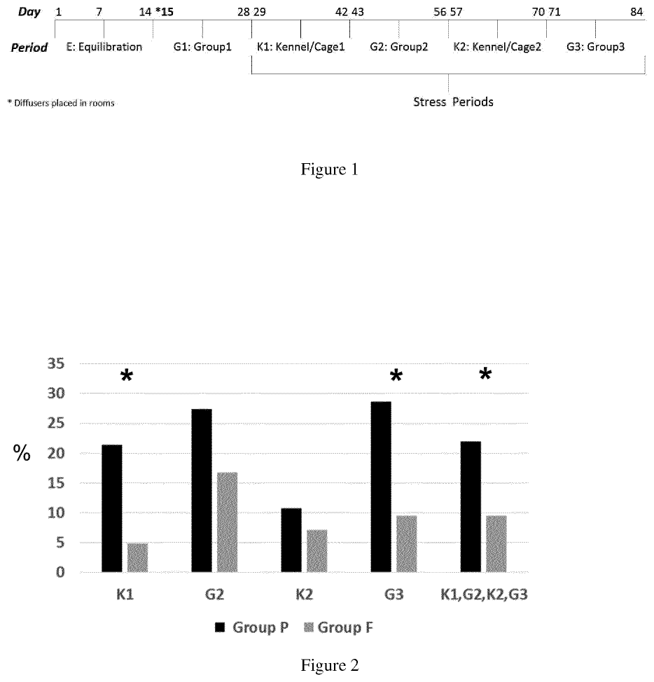

Experimental design. Two trained scorers, blinded regarding treatment allocation, applied the standardized clinical and behavioral scoring system at approximately the same time and order every morning, for 45 minutes per room, throughout the study (FIG. 1).

Assays. At the beginning of the study and after each of the six periods (E, G1, K1, G2, K2, G3), kittens were sedated, proparacaine was applied to their corneas, and blood, caudal pharynx mucosal cells, and conjunctival swabs were collected. Sera, oropharyngeal, and conjunctival swabs were stored at −80° C. until assayed in batches. Total DNA was extracted from the oropharyngeal and conjunctival swabs, and quantitative PCR assays (qPCR) were performed for FHV-1 DNA and GAPDH. Results of the FHV-1 qPCR assay were presented as the ratio of FHV-1 DNA/GAPDH DNA. Serum cortisol concentrations were measured at a commercial laboratory (Endocrinology Laboratory, Michigan State University).

### Statistical Evaluation

After randomization but before starting the study, the total clinical scores associated with FHV-1 that developed after primary infection in the previous study were compared between the pheromone group and the placebo group by Wilcoxon rank-sum test and the Groups were found to be similar (p=0.9).

Descriptive statistics were calculated and categorical data were expressed as frequencies, while continuous data was expressed as means, medians and ranges. Group median results for total clinical score (Table 1), individual clinical parameters, individual behavioral scores, kitten total stress scores, sleep scores, FHV-1/GAPDH ratios, body weights were compared between the pheromone group and placebo group by Wilcoxon rank-sum test.

After variables were screened for associations using the univariate analyses, if the p-value was <0.25, the variables were included in multivariable regression analyses. Multilevel mixed-effects logistic regression was used to assess the effect of individual kitten variation on results of clinical (binomial) and behavioral scores (binomial), and multilevel mixed-effects linear regression was used for total clinical scores and total stress scores. Group mean cortisol concentrations were compared between groups by two tailed Student's T test.

Commercially available software (STATA reference) were used for all comparisons. Significance was defined as p<0.05.

### Results

Clinical findings. All kittens had healthy appetites, gained weight consistently, and had soft, groomed fur each day throughout the study. None of the cats required medical intervention for FHV-1 infection. For 1 of 12 kittens, the temperature sensing microchip malfunctioned and so the body temperature in this kitten was measured in the axillary space. Since there were no manipulations to the kittens in G1 other than adding the diffusers, this was not considered a stress period for the final comparisons between groups: only the results from periods K1, G2, K2, and G3 were evaluated individually and in combinations. At the end of the equilibration period, there were no statistically significant clinical differences between groups (p=0.58). Mean, range, and group comparison results for the total clinical score listed by groups and study period in Table 3.

TABLE 3
Mean, range, and group comparison results for the total clinical scores by
group and study period.

| | Periods | | | | | |
|---|---|---|---|---|---|---|
| | G1 | K1 | G2 | K2 | G3 | K1, G2, K2, G3 |
| | Total score | | | | | |
| | Mean (range) | Mean (range) | Mean (range) | Mean (range) | Mean (range) | Mean |
| Placebo group | 13.7 (4-31) | 15.3 (2-29) | 16.2 (7-36) | 15.0 (3-33) | 14.8 (4-34) | 61.3 |
| Pheromone group | 12.8 (4-31 | 15.0 (2-28) | 12.0 (3-28) | 16.0 (5-29) | 13.2 (4-31) | 56.1 |
| Wilcoxon p value | 0.58 | 0.92 | 0.06 | 0.66 | 0.32 | 0.23 |

G2, G3 = the two, 2-week group housing periods after placement of the diffusers in G1;
K1, K2 = the two, 2-week kenneled periods.
The means and ranges are shown to demonstrate variation between the 6 kittens per group, but the medians are compared by Wilcoxon rank sum test for statistical evaluations with p < 0.05 being significant.

Coughing was rarely heard during the study. Serious ocular discharge was recorded at many observation points for multiple cats before and during the stress periods but was never associated with conjunctivitis. Serious nasal discharge was recorded frequently, but mucoid discharge associated with nasal congestion was not detected during the study. Thus, coughing, ocular discharge, and nasal discharge were not evaluated further. Mild nasal congestion (score=1) was reported commonly but moderate congestion was rarely reported and so this parameter was not evaluated further. Sneezing was the most common findings that were likely associated with FHV-1 infection. After the diffusers were placed during G1 but prior to the induction of stress, there was no difference between groups in sneezing (Placebo group=32.1%; Pheromone group=25.0%).

After controlling for individual kitten effects, sneezing occurred less frequently for kittens in the Pheromone group during period K1 (p=0.006), period G3 (p=0.005), and the combined stress periods (p<0.001).

### Behavioral Parameters

Based on review of the literature and agreement by the clinical scorers at the end of the study scoring periods, four behaviors were believed to be objective indicators of feline stress in this population (Table 4).

TABLE 4
Behaviorally relevant stress-related scores.

| Behavior | Time-period evaluated |
|---|---|
| Cage pacing | SS1, SS3, SS4*, Long |
| Cage pawing | SS1, SS3, SS4, Long |
| Cage disarray | Long |
| Excessive meow | Long |

*See Table 2 for a further description of how the behavior was determined and scored

A number of differences were detected between the 2 groups during the kenneled periods (Table 5). After controlling for individual kitten effect, more frequent cage pacing in placebo group kittens approached statistical significance during the SS3 time-point (p=0.05).

TABLE 5
Frequency of potential stress-related behaviors during time-points within
kenneled periods.

| | | | | | | If Wilcoxon rank-sum |
|---|---|---|---|---|---|---|
| | | | | | | <0.25, then mixed |
| | | | | | Wilcoxon rank-sum in | effects logistic |
| Time-period | Behavior | Group | K1 | K2 | K1 + K2 combined | regression |
| SS1 | Cage Pacing | Placebo | 52% | 33% | p = 0.69 | n/a |
| | | Pheromone | 45% | 36% | | |
| | Cage Pawing | Placebo | 21% | 11% | p = 0.09 | p = 0.19 |
| | | Pheromone | 11% | 7% | | |
| SS3 | Cage Pacing | Placebo | 7% | 1% | p = 0.02, Group P higher | p = 0.05, Group P higher |
| | | Pheromone | 2% | 0% | | |
| | Cage Pawing | Placebo | 6% | 1% | p = 0.43 | n/a |
| | | Pheromone | 10% | 1% | | |
| LONG | Cage disarray | Placebo | 44% | 48% | p = 0.004, Group P | p = 0.44 |
| | | Pheromone | 36% | 25% | higher | |
| | Cage Pacing | Placebo | 25% | 6% | p = 0.10 | p = 0.12 |
| | | Pheromone | 17% | 2% | | |
| | Cage Pawing | Placebo | 35% | 14% | p = 0.11 | p = 0.12 |
| | | Pheromone | 30% | 5% | | |
| | Cage Meow | Placebo | 57% | 37% | p = 0.04, Group P higher | p = 0.57 |
| | excessive | Pheromone | 43% | 29% | | |
| SS4 | Cage Pacing | Placebo | 0% | 0% | n/a | n/a |
| | | Pheromone | 2 | 0 | | |
| | Cage Pawing | Placebo | 5% | 0% | p = 0.52 | n/a |
| | | Pheromone | 7% | 0 | | |

n/a=not applicable

A total stress score for each kitten was calculated by adding the 4 stress-related behavior scores within the K1 and K2 stress periods; the total stress score was then compared between Groups. The pheromone group had significantly lower total stress scores than placebo group (Table 6). However, after controlling for individual kitten effect, the total kitten stress scores were not statistically different between Groups.

TABLE 6
Medians and ranges of the total stress score in the placebo
group and the pheromone group during the kenneled periods.

| | K1 Median | K2 Median | K1 and K2 Combined |
|---|---|---|---|
| | (range) | (range) | Median (range) |
| Placebo | 36 (19-52) | 19 (6-45) | 55 (26-97) |
| group | | | |
| Pheromone | 25 (19-46) | 16 (4-27) | 42 (23-73) |
| group | | | |
| p-value | 0.04 | 0.04 | 0.004 |

Median represents the median value of the 6 total stress scores per kitten per period.

Range represents the lowest (minimum) of the 6 total stress scores among the 6 kittens and the highest (maximum) total stress score among the 6 kittens in each Group;

Sleep was used as a correlate for “calm/relaxed” behavior, as it was observed and recorded at the end of the observation period at the SS4 time-point, after the room gradually quieted subsequent to the previous 45 minutes' activities. At that last time-point, if the kitten had a relaxed body posture and closed eyes, “sleep” was recorded. During the equilibration period, none of the kittens in pheromone group or placebo group had any occurrences of sleeping (Table 7).

TABLE 7
Proportions of cats sleeping at the end of each observation period

| | Equilibration | G1* | K1 | G2 | K2 | G3 |
|---|---|---|---|---|---|---|
| Placebo | 0% | 0% | 0% | 0% | 0% | 1% |
| group | | | | | | |
| Pheromone | 0% | 2% | 10% | 4% | 13% | 20% |
| group | | | | | | |

*diffusers placed at the start of G1

During study period G1, there were only two occurrences of sleeping in the pheromone group and no occurrences of sleeping in the placebo group. The pheromone group had significantly more sleeping events when the K1 and K2 stress periods were combined (p<0.0001) and when all 4 of the stress periods (K1, G2, K2, and G3) were combined (p<0.0001). After controlling for individual kitten effect in the multilevel mixed-effects logistic regression analysis, the pheromone group had significantly more sleeping only when all 4 of the stress periods were combined (p=0.002).

Other potential behavioral indicators of stress occurred infrequently in this kitten population. Isolated hissing events occurred between kittens during group play with novel objects on four occasions in both groups during period E, twice in both groups during period G1, and twice in the pheromone group and on six occasions in the placebo group during G2 and G3 combined. Similarly, fighting occurred during group play with novel objects, twice during period E and once during period G1 in the pheromone group, and it occurred on four occasions during period G2 in the placebo group. Diarrhea was reported once in three kittens and four times in one kitten throughout the study period. When group housed, all kittens greeted scorers at the door daily. Hiding was not observed among the kittens, nor did any kittens show any aggressive or fearful behaviors during the scoring periods.

Cortisol results. All serum cortisol concentrations were in the normal range reported by the laboratory. The mean results for the pheromone group and the placebo group did not differ over the course of the study. However, the mean cortisol results were greater at the end of the last stress period (G3?) when compared to the start of the first stress period (K1), for Pheromone group (p=0.046), Placebo group (p=0.04), and the 2 groups combined (p=0.007).

FHV-1/GAPDH ratios. While GAPDH DNA was amplified from almost all of the swabs collected from the kittens, FHV-1 DNA was only amplified for? 2 kittens in the placebo group and 4 kittens in the pheromone group after starting the first stress period (K1). The number of samples with detectable FHV-1/GAPDH ratios was too small to allow for comparisons in viral shedding magnitude between groups. When the group mean cortisol results from the end of the last stress period were compared between kittens that did or did not shed FHV-1 during the stress periods, there were no statistical differences (p=0.125).

### Discussions

Even when a standardized dose of FHV-1 is used to inoculate kittens born from FHV-1 naïve queens, there are variations in the clinical signs of disease. In addition, collection of objective data to assess behavior in cats can be difficult and individual cats respond to stress differently. Reactivation of FHV-1 associated illness or disorders in response to stress may not happen in some cats and when reactivation does occur, time until recognition of clinical signs can vary from 4-11 days. Thus, combining the results from different stress periods may be the most accurate way to evaluate for differences between groups in this study. The authors believe the biggest limitation to this pilot study was the inclusion of only 6 cats per group which may have lessened the chances of detecting statistically significant differences between groups. However, after controlling for individual kitten effect and combining the 4 presumed stress periods, kittens in the room with the pheromone diffuser had increased sleeping at the end of the observation periods and overall less sneezing throughout the periods when compared to those kittens in the room with the placebo diffuser, which supports a treatment effect.

This stress model to attempt to reactivate FHV-1 has only been used in one study of a probiotic with presumed immunomodulating activity. Similar to the results described in the current study, evidence for reactivation of FHV-1 varied amongst cats but a treatment effect was documented. In the previous study, conjunctivitis was common and sneezing was rare whereas in the study described here, conjunctivitis was rare but sneezing was common. These differences between studies were likely from the use of 2 different FHV-1 strains and inoculation methods. In the previous study, the field strain of FHV-1 was administered into the conjunctiva fornix whereas the different FHV-1 strain used in the study described here was administered by nasal inoculation.

In this study, coughing was rare, serous ocular discharges were not associated with conjunctivitis, mucoid nasal discharges did not occur, and moderate nasal congestion was only rarely reported, and so were not evaluated individually. However, sneezing is an objective finding and having 2 scorers present at one time made it possible to determine which kittens sneezed during the observation periods. All kittens still had intermittent sneezing from the primary FHV-1 infection as they entered this study and sneezing was still common in the placebo group (32.1% of the time) and the pheromone group (25.0%) during G1 when the diffusers were first placed. However, over time in the 4 presumed stress periods, the cats in the room with the pheromone diffuser had generally decreasing proportions of observation points with sneezing whereas cats in the room with the placebo diffuser maintained proportions of observation points with sneezing similar to the time periods prior to the presumed induction of stress (FIG. 2). These findings could indicate reactivation or maintenance of FHV-1 associated sneezing in the placebo room, presumably because of a greater receptivity to stress exposure. If a study like this is performed again in the future, splitting the FHV-1 inoculum between the nose and eyes may better reflect a natural infection and make it more likely to be able to recognize reactivation of disease.

GAPDH was amplified from almost all of the oropharyngeal swabs and conjunctival swabs suggesting that the sample collections were adequate. However, FHV-1 DNA was rarely amplified from these sample sites and times. As FHV-1 is not eliminated after inoculation, it is likely that many of the FHV-1 PCR assay results are falsely negative. Multiple studies have shown that FHV-1 PCR assays results can be negative even in the presence of disease as infectious organism numbers are suppressed by the immune responses. Use of small biopsies of the conjunctiva may be more sensitive than use of swabs and this should be considered for use in future studies.

Another limitation was at the inception of this study, there was no previously published scoring rubric for studying stress in well adapted research kittens. Thus, in this pilot study the inventors attempted to design their own behavioral metric in order to assess these particular kittens that had unique personalities and experiences, of which the investigators and observers were aware prior to the start of the study (Table 2). The kittens were already well socialized, affectionate, attention-seeking of people, and were habituated to the research environment, each other, and the observers. All kittens accepted and often sought gentle handling and human contact, petting, and play with each other and the observers and the observers' scoring implements and attire. Therefore, the typical indicators of stress and fear such as hiding, freezing, stiffness, crouching, hissing, dilated pupils, and holding ears back could not be evaluated in this study because those behaviors were not displayed by these particular kittens. And so even though data was collected on a total of 28 behaviors recorded during multiple time-points, only cage pacing, cage pawing, cage disarray and excessive meowing (Tables 4 and 5) could be evaluated objectively for statistical comparisons between groups. The behavioral scoring rubric that was ultimately used might not have captured sufficient data to assess overall stress or relaxation, as we only used one 45-minute time period and did not observe the kittens for the other 23 hours of the day. Therefore, although the cage-stress period clearly resulted in acute stress in the kittens while they were being observed, we do not know the kittens' behaviors for the other 23 hours of the day. It is possible that they were not exhibiting stress behaviors, thus not experiencing prolonged stress, while the observers were not in the room. All kittens retained well-groomed fur, all ate and gained weight appropriately, and no relevant diarrhea nor vomiting was observed.

While cage pacing, cage pawing, cage disarray and excessive meowing were usually numerically greater in the room with the placebo diffuser, after accounting for individual kitten variability, increased cage pacing in the placebo diffuser group at only one time point approached statistical significance (Table 5). In addition, while the total stress scores were significantly greater in the cats in the placebo diffuser room during each kenneled period and the kenneled periods combined in the univariate analysis, this difference was lost when analyses were adjusted for individual kitten variations. It is possible that this failure to find differences between groups in the multivariate analysis was merely due to limited sample size.

Stress amount, type, frequency, and duration necessary to reactivate FHV-1 are still unclear. Upper respiratory tract disease (URTD) is a frequent finding in shelters, and the probability of having URTD gradually increases as length of stay in the shelter increases. One study found that over 80% of cats developed URTD by day 14 in the shelter. However, shelters expose cats and kittens to a multitude of variables, including stress and collective environment, which likely concomitantly contribute to URTD and clinical signs. Our study mainly attempted to alter stress by housing change and venipuncture to assess the effect on FHV-1 recrudescence.

While all cortisol concentrations remained in the normal range from the reference laboratory, there was a significant difference between the pre-stress period and end of study cortisol concentrations in both groups of cats, suggesting stress had occurred over time. In addition, there were other events in the study than housing changes that were likely associated with stress including blood collection, witnessing other kittens in distress during blood collection, and occasional variations in feeding times.^{11,2352-55}. In addition, the kittens in the placebo group were always scored first, and it is possible that the kittens in the pheromone diffuser group could hear the kitten-human interactions leading to an additional 45 minutes of arousing, possibly stressful. These potential confounding factors should be addressed in future studies.

It is possible that differences among groups in many measures were due to the small sample size which would be affected by individual differences in stress responses among the kittens. Individuals experience wide variations in stress responses, even when exposed to the same stress. Animals differ not only in their outward behavioral response to the stress, but also in their personality, neurophysiologic and immune response.²⁰ Thus individual variations in our kittens' responses to stress could have also resulted in individual variations in reactivation of FHV-1 and outward clinical signs, as coping mechanisms and coping efficiencies differ between individuals.

In the kittens of this study, the best indicator of a relaxed state was considered to be sleeping at the end of the 45 minute observation period (SS4). The authors believe that the SS4 time-point served as a reliable indicator for true sleeping, as it recorded behaviors after the kittens were accustomed to the observers having been in the room for 45 minutes, after the room gradually quieted subsequent to other activities. Sleeping did not occur during the equilibration period, during times of activity in the rooms including scoring, when observers first entered the room or if facility noise was audible outside of the room. Furthermore, during each sleeping occurrence, the kitten had a relaxed body posture and closed eyes. When the 45-minute observation time concluded, and the observers arose from the floor to depart the room, all kittens awakened, sometimes stretched, and actively sought attention again from the observers. This also supports our assessment of this behavior as an indicator of a truly relaxed state. Sleeping was observed with gradual increased frequency in the room with the pheromone diffuser after the diffusers were in place (Table 7). “Feigning sleep” has been used as an indicator of stress in cats, particularly shelter and caged cats and it has been described as a defensive sleeping posture in captive felids in zoos, for example. However, it appeared here that none of them represented feigned sleep.

Conclusions. Taken in total, the data support reactivation or maintenance of sneezing associated with FHV-1 in the kittens in the placebo diffuser room as opposed to a significantly decreased occurrence of sneezing in the kittens exposed to the pheromone composition. The evidence for decreased stress and increased relaxation evidenced by increased sleeping at the end of the observation period in the kittens in the pheromone diffuser room compared to the placebo diffuser room supports the hypothesis that exposure to the pheromone lessened stress and sneezing associated with FHV-1.

1-15. (canceled)

16. A method for treating feline chronic respiratory disorders or complications thereof and/or feline chronic infectious disorders or complications comprising administering to a feline in need thereof a veterinary composition comprising pheromones.

17. The method according to claim 16, wherein the feline has a feline chronic respiratory disorders.

18. The method according to claim 16, wherein the feline chronic infectious disorders or feline chronic respiratory disorders are due to infections.

19. The method according to claim 16, wherein the feline chronic respiratory disorders is caused by a herpes infection or a FHV-1 infection.

20. The method according to claim 16, wherein the feline chronic respiratory tract disorder is a chronic upper respiratory tract disorders.

21. The method according to claim 20, wherein the treatment lessens or reduces sneezing.

22. The method according to claim 16, wherein said treatment increases relaxation as evidenced by increased sleep period of felines with infectious disorders and/or respiratory disorders.

23. The method according to claim 18, said method reducing clinical symptoms due to feline chronic infections.

24. The method according to claim 23, wherein said clinical symptoms due to feline chronic infections are cough, sneezing, nasal discharge, nasal congestion, ocular discharge, ocular congestion, and/or blepharospasm.

25. The method according to claim 17, said method reducing clinical symptoms due to feline chronic respiratory disorders.

26. The method according to claim 25, wherein said clinical symptoms due to feline chronic respiratory disorders due to infections are cough, sneezing, nasal discharge, and/or nasal congestion.

27. The method according to claim 16, wherein the veterinary composition comprises a mixture of oleic acid, azelaic acid, pimelic acid and palmitic acid, or derivatives thereof.

28. The method according to claim 16, wherein the composition comprises at least a therapeutically effective amount of active fatty acids or ester or methyl ester derivatives thereof, said fatty acids being selected from the group consisting of oleic acid, azelaic acid, pimelic acid and palmitic acid.

29. The method according to claim 16, wherein the composition comprises a mixture of fatty acids comprising at least about 45-60% oleic acid, 6-10% of azelaic acid, 8-12% of pimelic acid, and 13-18% of palmitic acid, with respect to the total weight of fatty acids and derivatives thereof, or a mixture comprising between about 45-65% methyl oleate, between about 6-10% dimethyl azelate, between about 8-12% of dimethyl pimelate, and between about 13-18% of methyl palmitate, with respect to the total weight of fatty acids and derivatives thereof, or a mixture of esters of fatty acids comprising approximately 47-51% of methyl oleate, approximately 7-9% of azelate of dimethyl, between 9-11% of dimethyl pimelate and approximately 14-16% of methyl palmitate, with respect to the total weight of fatty acids and derivatives thereof.
